(19) United States
(12) Patent Application Publication
(10) Pub. No.: US 2024/0347172 A1
(43) Pub. Date: Oct. 17, 2024

(54) SYSTEMS AND METHODS FOR IDENTIFYING RELEVANT STUDIES

(71) Applicant: Optum, Inc., Minnetonka, MN (US)

(72) Inventors: Sara Daneshvar, Port Moody (CA); Paul Alain Vial, Vancouver (CA); David Dubois, Mirabel (CA)

(21) Appl. No.: 18/301,697

(22) Filed: Apr. 17, 2023

(51) Int. Cl. G16H 30/40

(52) U.S. Cl. CPC G16H 30/40

(57) ABSTRACT

In order to improve medical care, systems and methods for identifying relevant studies are provided. An A.I. model, such as a machine learning model, is trained to identify findings associated with studies based on the text of reports included in the studies. Later, when a medical professional is viewing a current study for a patient in a PACS viewer or application, the model is used to determine findings associated with previous studies associated with the patient. These determined findings are displayed to the medical professional in the PACS viewer. The medical professional may then select a finding that she may think is relevant to the current study. In response, one or more previous studies associated with the selected finding may be displayed to the medical professional in the PACS viewer including portions of the associated reports or images from the previous studies.

### BACKGROUND

Picture Archiving and Communication Systems (“PACS”) are used by medical professionals to view studies for patients. A study for a patient may include images or videos of the patient and may include X-Rays, CAT scans, MRIs, and other medical images or videos. A study may further include a report that includes other information collected from the patient such as symptoms, vital signs, current medications, and other observations of the medical professional. The report may also include one or more diagnoses or conclusions of the doctor or medical professional, as well as a course of action or recommended medications for the patient.

Generally, a medical professional views a study for a patient using a PACS viewer or viewing application. The medical professional may use the viewing application to select the images from a study to view, to review the associated report, or to enter new or updated information into the report.

While PACS and PACS viewing applications are useful, there are associated drawbacks. Once such drawback is that for a medical professional viewing a current study for a patient and trying to determine a diagnosis and course of treatment for a patient, there is no easy way for the medical professional to quickly and easily search diagnoses or findings in the bodies of reports associated with past studies for the patient. This is because medical professionals often use non-standard terminology when entering text into reports making it difficult to search the reports without knowing the exact phrasing and terminology used by the authors of the reports. In addition, reports are typically comprised of unstructured data, which are less searchable than encoded database fields, for example.

### SUMMARY

In order to improve medical care, systems and methods for identifying relevant studies are provided. An A.I. model, such as a machine learning model, is trained to identify findings associated with studies based on the text of reports included in the studies. Later, when a medical professional is viewing a current study for a patient in a PACS viewer or application, the model is used to determine findings associated with previous studies associated with the patient. These determined findings are displayed to the medical professional in the PACS viewer. The medical professional may then select a finding that he/she may think is relevant to the current study. In response, one or more previous studies associated with the selected finding may be displayed to the medical professional in the PACS viewer including portions of the associated reports or images from the previous studies. The model can further be used to filter studies in an educational system or to generate statistics related to certain diagnoses or findings.

The systems and methods for identifying relevant studies described herein provide the following advantages. First, by normalizing the findings associated with reports to a standard terminology or set of terms, the associated studies can be quickly filtered and searched to identify relevant studies. Relevant past studies for a current patient can be used for a variety of purposes including guiding or informing a current diagnosis. Second, because the findings for a report are identified using a model, rather than by a medical professional, the human labor associated with normalizing or determining findings is greatly reduced.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying figures, which are incorporated herein and form part of the specification, illustrate a system and method for determining findings for medical studies using artificial intelligence in order to more efficiently identify relevant studies. Together with the description, the figures further serve to explain the principles of the system and method described herein and thereby enable a person skilled in the pertinent art to make and use the system and method for determining findings for medical studies using artificial intelligence.

FIG. 1 is an example environment for identifying relevant studies;

FIG. 2 is an illustration of an example method for training a model to determine findings associated with reports and studies;

FIG. 3 is an illustration of an example method for displaying relevant prior findings, reports, and studies;

FIGS. 4-6 are illustrations of an example user interfaces for displaying relevant prior findings, reports, and studies; and

FIG. 7 shows an exemplary computing environment in which example embodiments and aspects may be implemented.

### DETAILED DESCRIPTION

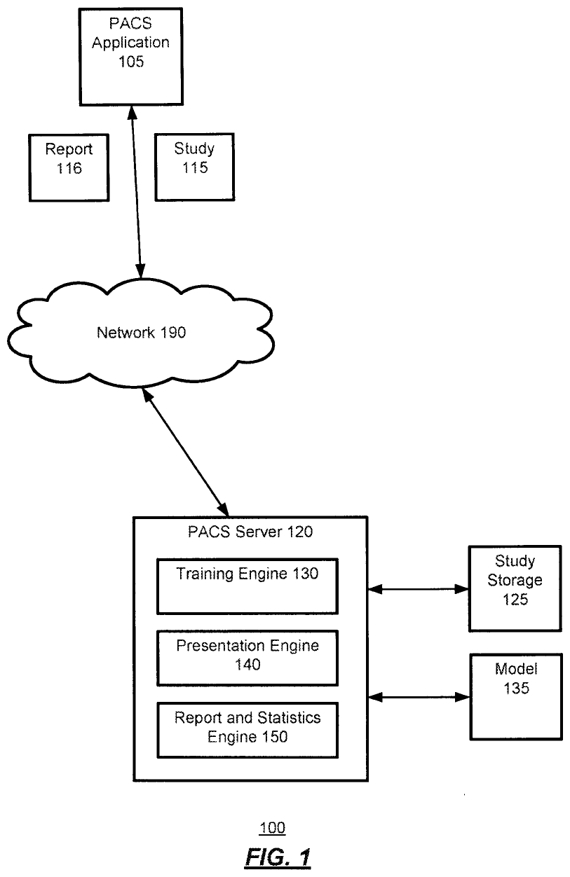

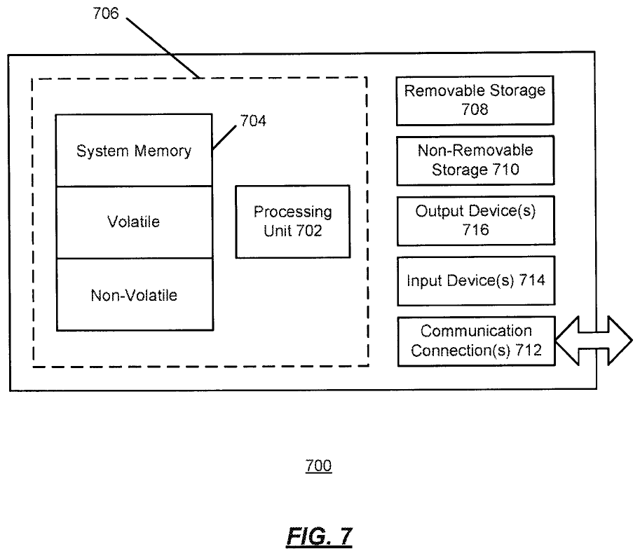

FIG. 1 is an example environment for identifying relevant studies. As shown, the environment 100 includes a PACS application 105 in communication with a PACS server 120 through a network 190. The network 190 may include a combination of private and public networks (e.g., the internet). The PACS server 120, and the PACS application 105 may each be executed by one or more general purpose computing devices such as the computing device 700 illustrated with respect to FIG. 7.

Note that while the PACS application 105 and PACS server 120 are shown as separate from one another, this is for illustration only. In some embodiments, the PACS application 105 and PACS server 120 may be implemented together as a single application and/or may be executed by the same computing device.

The PACS application 105 may be an application used to view and interact with one or more studies 115. The studies 115 may be medical studies and each may include metadata and a plurality of objects. The metadata may include information about the study 115 such as the order of objects in the study 115, and the types of object in the study 115 for example. Other information may be included in the metadata.

The objects of the viewing study 115 may include a variety of object types such as image objects, and Grayscale Softcopy Presentation State (“GSPS”) objects. Other types of objects may be included such as presentation information (e.g., annotations, styles, flagged images/frames), relevant academic/references scans, documents, and any other information that may be linked to the aforementioned entities.

With respect to image objects, these may include a variety of medical images and DICOM image objects including X-ray images, CT scan images, and MRI images. Other types of images may be included. Typically, each study 115 may include a variety of views and each view may be associated with multiple image objects. In addition, the image objects may include thumbnail images that are of a lower resolution than the images that they represent.

Each study 115 may be associated with one or more reports 116. Depending on the embodiment, a report 116 for a study 115 may include text entered or dictated by a medical professional who viewed the study 115. The report 116 may include information such as observations of the medical professional regarding the patient associated with the study, lists of medications taken by the patient, and vital signs of the patient (e.g., temperature and blood pressure). Also associated with the report 116 may be one or more findings made by the medical professional for the patient based on the study 115. As used herein findings may include any finding, including incidental findings, made by the medical professional while viewing the study 115 such as any diagnoses or observed symptoms.

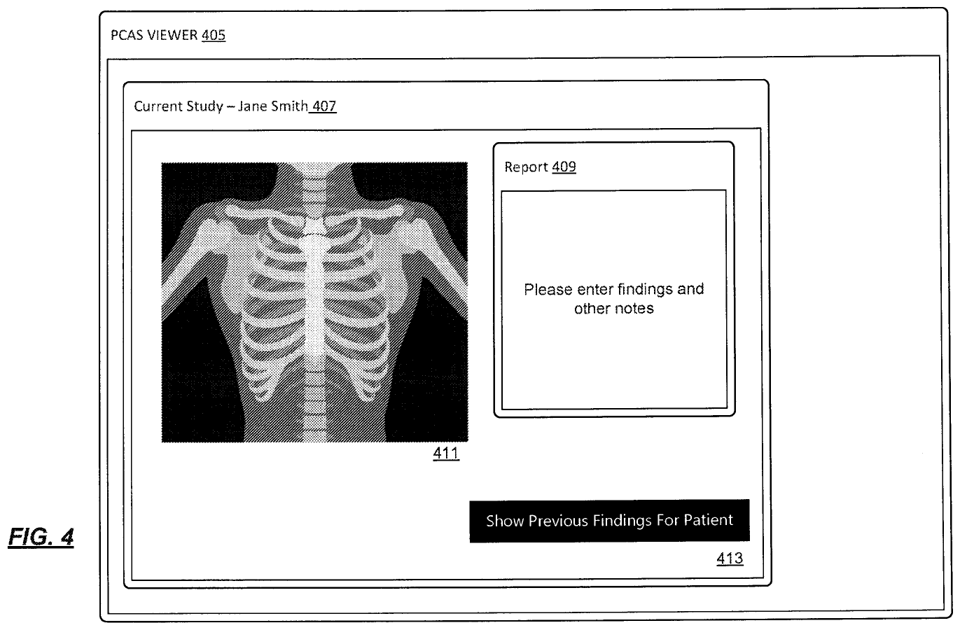

The PACS server 120 may store one or more studies 115 (including associated reports 116 and objects) in study storage 125. When a medical professional (or other user) desires to view a study 115, the PACS application 105 may send a request for the study 115 to the PACS server 120. In response, the PACS server 120 may retrieve the requested study 115 (including report 116), and may send them to the PACS application 105 via the network 190. The PACS application 105 may then render and display the study 115 and report 116 to the medical professional in a graphical user interface. As will be described further below, an example graphical user interface is shown in FIGS. 4-6.

As described above, one drawback associated with PACS applications 105 is that there is no easy way to search reports 116 to identify relevant studies 115 based on findings. For medical providers, this may result in the providers having to review multiple possibly irrelevant studies 115 associated with a patient to uncover relevant past findings for the patient, which is a waste of time and resources for already overburdened providers. To solve this problem and others, the PACS server 120 may further include a training engine 130. The training engine 130 may use training data to generate a model 135 that, based on the text of a report 116, outputs one or more findings associated with the report 116. The output findings may include one or more diagnoses, for example.

Depending on the embodiment, the model 135 may output findings that are related to the findings in the report 116, but that may not be explicitly mentioned in the report. For example, a report 116 may have a finding of “Glioblastoma,” which is a type of tumor. The model 135 may associate the finding of “Glioblastoma” with the report 116 because it was mentioned by the medical professional. However, the model 135 may also associate the finding of “tumor” with the report because it will be useful for searching later, even though it was not explicitly mentioned by the medical professional in the report 116.

In order to bring uniformity to the findings, the findings output by the model 135 may be selected from a set of standardized medical terminology. In one example, the findings may be selected from the International Classification of Diseases 10th Revision (ICD-10). Other references may be used.

The training engine 130 may train the model 135 using a set of training data. In some embodiments, the training data may include a set of reports 116 where each report 116 has been labeled with one or more findings. The findings may be taken from the ICD-10, for example. Depending on the embodiment, each report 116 may be labeled by one or more human reviewers. Other methods for generating training data may be used.

In some embodiments, the model 135 may be an NLP-based model 135 and may be trained using machine learning. Example models 135 include neural networks. Other types of models 135 may be supported.

After generating the model 135, the training engine 130 may use the model 135 to generate one or more findings for each study 115 in the study storage 125 based on the report 116 associated with each study 115. The generated findings may be stored along with the studies 115 in the study storage 125.

As may be appreciated, once the findings have been associated with the studies 115, the studies 115 and their uniform findings can be used for a variety of new and useful applications. One such application is study 115 searching and filtering by finding.

To facilitate the searching and filtering, the PACS server 120 further includes a presentation engine 140. In one embodiment, the presentation engine 140 may allow a medical professional to filter or search previous studies 115 for a patient based on the findings associated with each of the studies 115. For example, a medical professional may be treating a patient for a possible tumor. To assist in her diagnosis, the medical professional may desire to see all of the previous studies 115 associated with the patient with a finding of tumor. The medical professional may enter the query tumor into a search field provided by the PACS application 105, and in response to the query, the presentation engine 140 may provide links to any study 115 associated with the patient that has a finding of tumor. The PACS application 105 may then present the links to the medical professional.

In another example, rather than the medical professional providing the query, when the medical professional is viewing a current study 115 for a patient, or in response to selecting the patient, the presentation engine 140 may provide a list of some or all of the findings associated with previous studies 115 associated with the patient. The list may be presented based on the frequency that each finding is associated with a study 115 or based on some other criteria like the body region of the current study, for example. The medical professional may then select the finding that they are interested in, and links to one or more corresponding studies 115 may be provided to the medical professional by the presentation engine 140 through the PACS application 105.

For example, FIGS. 4-6 are illustrations of example user interfaces of a PACS application 105 incorporating some of the above-described features. Starting at FIG. 4, a medical professional is using the PACS application 105 to view a study for a patient named “Jane Smith.” The PACS application 105 includes a user interface window 405 that displays the current study 115 in a sub-window 407. As shown in the sub-window 407 is an X-Ray 411 from the current study 115, and a text box 409 where the medical professional can enter findings and other text for the corresponding report 116.

Also shown is a user interface element 413 with the text “Show Previous Findings For Patient.” The medical professional may select the element 413 to view the findings determined for previous studies 115 stored for the patient in the study storage 125.

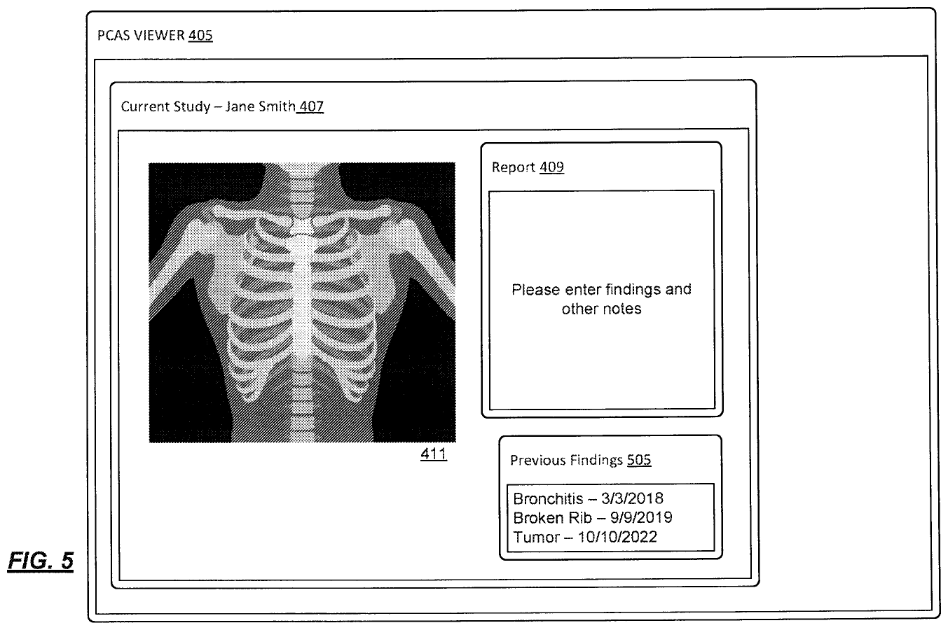

Continuing to FIG. 5, the medical professional has selected the user interface element 413, and in response the presentation engine 140 has returned a list of previous findings that are displayed in a sub-window 505. As shown, each finding includes a date of the most recent study 115 associated with the finding. The medical professional may select one of the findings in the sub-window 505 to view the study 115 associated with the finding.

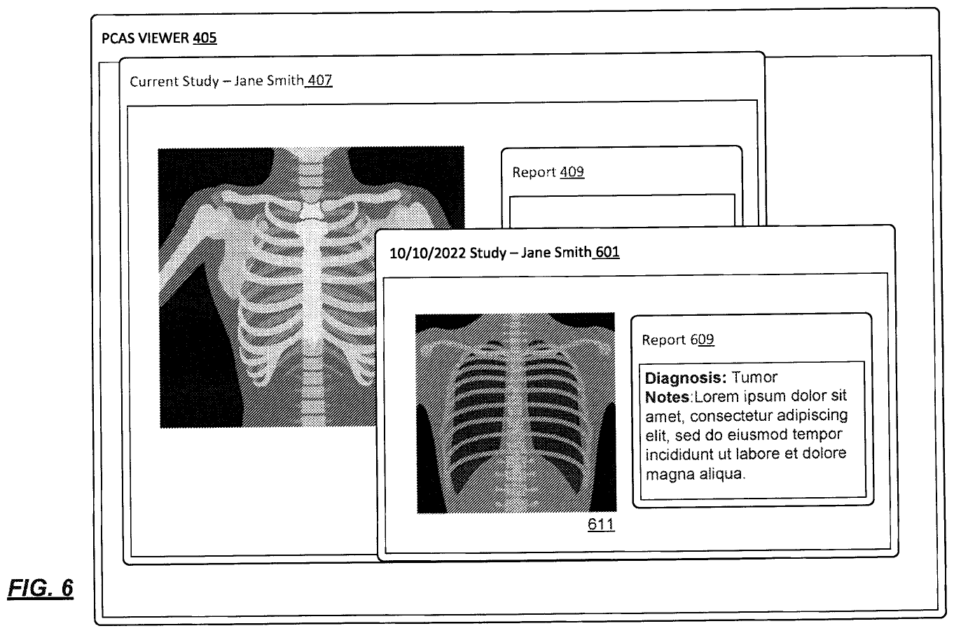

Continuing to FIG. 6, the medical professional has selected the finding “Tumor” in the sub-window 505, and in response, a window 601 is displayed that shows the study 115 corresponding to the selected finding. The window 601 includes an X-Ray 611 from the study 115, and a report 609. The medical professional can view the study 115 and the report 116 in the window 601 (or another window such as the window 407, for example) to assist her in making her findings for the report 116 for the current study 115.

Having access to previous studies, findings, or diagnoses associated with a patient in the PACS application 105 has many benefits. One such benefit is in emergency scenarios where a patient may be unconscious or may otherwise be unable to communicate with a medical professional. The medical professional can see previous findings and diagnoses associated with the patient in the PACS application 105 which is more convenient than other methods for viewing the medical history of the patient.

Another benefit is that knowing previous findings for a patient can assist the medical professional in directing his/her focus when viewing a current study 115. For example, if the medical professional is made aware of a previous finding of Osteoporosis, she can look for signs of fractures in the current study 115. As another example, if a medical professional is made aware of a previous finding of a stroke, she can look for signs of clots in the current study 115.

Another benefit is that knowing previous findings can provide additional evidence for a current diagnosis. For example, a medical professional may not be confident whether she is looking for a tumor in a study 115. However, after seeing that tumor was a finding in a prior study 115 associated with the patient, she would gain confidence on her diagnosis by knowing that the patient had prior history of tumor in other body parts.

Another benefit of knowing previous findings is the early diagnosis of relevant health issues. For example, a medical professional may be viewing a study 115 that includes X-Rays of a patient after the patient suffered a car accident. Due to the car accident, the medical professional may have initially been looking for signs of fractures in the X-ray. However, after seeing that the patient has previous studies 115 associated with the finding of tumors, the medical professional may also look for new signs of tumors in the current study 115 along with fractures.

Returning to FIG. 1, the PACS server 120 may further include a report and statistics engine 150. In some embodiments, the report and statistics engine 150 may allow researchers or other users to search for studies 115 based on the associated findings. The report and statistics engine 150 may further allow the researchers to focus their search on certain dates, or based on certain patient demographics, locations, or other criteria. For example, a researcher may be studying heart disease among women between the ages of 35 and 45. Accordingly, the researcher may use the PACS application 105 to provide a query for studies 115 of women between the ages of 35 and 45 that are associated with the finding “heart disease.” The report and statistics engine 150 may then return links to all studies 115 associated with the finding “heart disease” that meet the filtering criteria.

In some embodiments, the report and statistics engine 150 can be used to search for studies 115 that are connected to PACS or not connected to PACS (i.e., studies 115 that have their own associated datasets that are not part of PACS). The researchers or users may select the particular datasets to include in their search when focusing their search.

In some embodiments, the report and statistics engine 150 may allow researchers or other users to generate statistics based on findings and other criteria associated with studies 115. For example, a researcher may be studying recent increases in pancreatic cancer in certain zip codes of the United States and may use the report and statistics engine 150 to generate counts of studies 115 associated with findings of pancreatic cancer for various zip codes for specified periods of time. The counts may be used to identify zip codes associated with decreases or increases in pancreatic cancer rates over the specified periods of time.

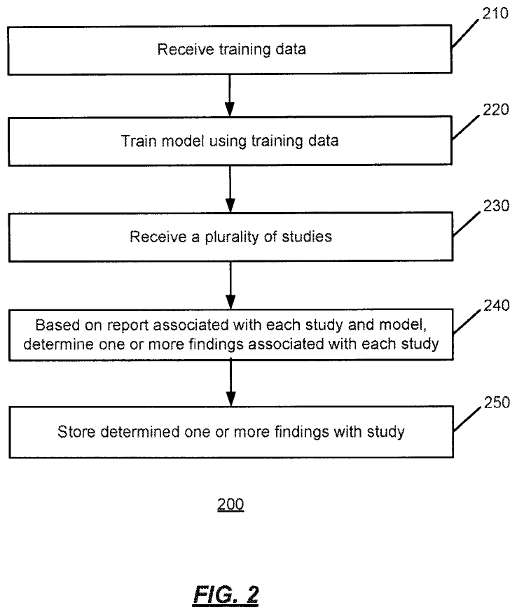

FIG. 2 is an illustration of an example method for training a model to determine findings associated with reports and studies. The method 200 may be implemented by the training engine 130 of the PACS server 120.

At 210, training data is received. The training data may be received by the training engine 130 of the PACS server 120. The training data may be labeled training data and may include a plurality of reports 116, with each report 116 labeled with one or more findings (e.g., diagnoses) by one or more human reviewers.

At 220, a model is trained using the training data. The model 135 may be trained by the training engine 130 of the PACS server 120. The model 135 may be trained using machine learning based on the text of each report 116 of the training data and the labels. The model 135 may be a neural network and may be trained to output or generate one or more findings based on an input report 116. Depending on the embodiment, a portion of the training data may be reserved for testing the findings predicted by the model 135 and for making any adjustments to the model 135 based on the predicted findings versus the labeled findings.

At 230, a plurality of studies is received. The plurality of studies 115 may be received by the training engine 130 from the study storage 125. The studies 115 of the plurality of studies 115 may be studies 115 that are associated with patients and may be associated with one or more reports 116.

At 240, one or more findings associated with each study are determined. The one or more findings for a study 115 of the plurality of studies 115 may be determined by the training engine 130 using the model 135 and the report 116 associated with the study 115. The stored studies 115 and determined one or more findings may be later used for a variety of purposes including filtering and searching by medical professionals, scientific research, and statistical analysis.

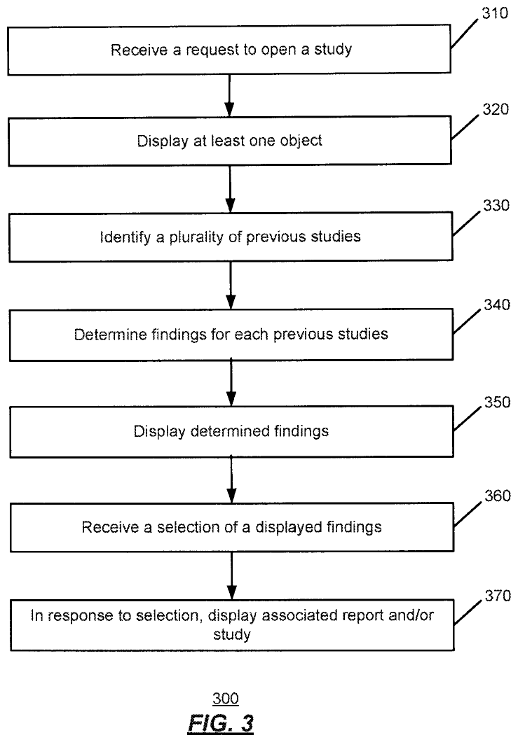

FIG. 3 is an illustration of an example method for displaying relevant prior findings, reports, and studies. The method 300 may be implemented by the presentation engine 140 of the PACS server 120.

At 310, a request to open a study is received. The request may be received by the presentation engine 140 of the PACS server 120 from a PACS application 105 being used by a medical professional. The request may be to open a study associated with a current patient of the medical professional. The study 115 may be a medical study 115 and may include a plurality of objects. Each object may be a medical image such as an X-ray, for example.

At 320, at least one object of the study is displayed. The at least one object may be displayed to the medical professional through a user interface provided by the PACS application 105. The medical professional may use the PACS application 105 to interact with the at least one object (and other objects of the study). The medical professional may further provide any findings to the PACS application 105 by entering or speaking text. The text may be saved with the study 115 as part of a report 116.

At 330, a plurality of previous studies associated with the patient is identified. The plurality of studies 115 may be stored in the study storage 125 and may be determined by the presentation engine 140 using an identifier of the patient.

At 340, findings for the previous studies are determined. The finding for the previous studies may be determined by the presentation engine 140. In some embodiments, each stored study 115 may have associated findings that were determined using a report 116 associated with the study 115 and a model 135 that was trained to determine findings for studies 115 based on reports 116. If no findings are associated with any of the identified previous studies 115, the presentation engine 140 may determine the findings using the model 135 and the reports 116 associated with the identified studies 115.

At 350, at least some of the determined findings are displayed. The at least some of the determined findings from the previous studies may be displayed to the medical professional in a user interface of the PACS application 105. The medical professional may view the displayed findings and may determine if any of the findings are helpful or relevant to the current study being viewed.

At 360, a selection of one of the displayed findings is received. The selection may be received by the presentation engine 140 from the PACS application 105. For example, the medical professional may have used a mouse (or other input device) to select one of the displayed findings.

At 370, in response to the selection, an associated report and/or study is displayed. The study 115 corresponding to the selected finding may be displayed to the medical professional by the PACS application 105. Alternatively or additionally, the report 116 associated with the study 115 may also be displayed. The study 115 and/or report 116 may be displayed in a same or different user interface as the original study 115 that was opened by the medical professional. Alternatively or additionally, indicators of multiple relevant reports 116 and/or studies 115 may be displayed to the medical professional, and the medical professional may then select a study 115 and/or report 116 to view.

FIG. 7 shows an exemplary computing environment in which example embodiments and aspects may be implemented. The computing device environment is only one example of a suitable computing environment and is not intended to suggest any limitation as to the scope of use or functionality.

Numerous other general purpose or special purpose computing devices environments or configurations may be used. Examples of well-known computing devices, environments, and/or configurations that may be suitable for use include, but are not limited to, personal computers, server computers, handheld or laptop devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 7, an exemplary system for implementing aspects described herein includes a computing device, such as computing device 700. In its most basic configuration, computing device 700 typically includes at least one processing unit 702 and memory 704. Depending on the exact configuration and type of computing device, memory 704 may be volatile (such as random access memory (RAM)), non-volatile (such as read-only memory (ROM), flash memory, etc.), or some combination of the two. This most basic configuration is illustrated in FIG. 7 by dashed line 706.

Computing device 700 may have additional features/functionality. For example, computing device 700 may include additional storage (removable and/or non-removable) including, but not limited to, magnetic or optical disks or tape. Such additional storage is illustrated in FIG. 7 by removable storage 708 and non-removable storage 710.

Computing device 700 typically includes a variety of computer readable media. Computer readable media can be any available media that can be accessed by the device 700 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 704, removable storage 708, and non-removable storage 710 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 700. Any such computer storage media may be part of computing device 700.

Computing device 700 may contain communication connection(s) 712 that allow the device to communicate with other devices. Computing device 700 may also have input device(s) 714 such as a keyboard, mouse, pen, voice input device, touch input device, etc. Output device(s) 716 such as a display, speakers, printer, etc. may also be included. All these devices are well known in the art and need not be discussed at length here.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although exemplary implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A method comprising:
displaying at least one object of a plurality of objects of a study associated with a patient in a user interface by a computing device;
identifying a plurality of previous studies associated with the patient by the computing device;
for each previous study of the plurality of previous studies, determining one or more findings associated with the previous study by the computing device;
displaying at least one of the determined one or more findings in the user interface by the computing device;
receiving a selection of the displayed at least one of the determined one or more findings by the computing device; and
in response to the selection, displaying the previous study associated with the selected at least one of the one or more determined findings in the user interface by the computing device.

2. The method of claim 1, further comprising:
in response to the selection, displaying one or more objects associated with the selected at least one of the one or more determined findings in the user interface.

3. The method of claim 1, wherein determining the one or more findings associated with the previous study comprises:
determining a report associated with the previous study;
retrieving a model trained to identify one or more findings based on a report; and
determining the one or more findings associated with the previous study using the model and the determined report.

4. The method of claim 3, further comprising:
receiving training data comprising a plurality of reports, wherein each report is labeled with at least one finding associated with the report; and
training the model using the training data by the computing device.

5. The method of claim 4, wherein the model comprises a machine learning model.

6. The method of claim 1, wherein the study comprises an image study.

7. The method of claim 1, wherein the objects comprise X-ray studies, CAT scan studies, ultrasound studies, positron emission tomography studies, nuclear medicine studies, mammography studies, or MRI studies.

8. The method of claim 1, wherein the one or more findings comprise one or more diagnoses.

9. The method of claim 1, wherein the at least one object is displayed in response to receiving a request to open the study.

10. A system comprising:
at least one computing device; and
a computer-readable medium with computer-executable instructions stored thereon that when executed by the at least one computing device cause the system to:
display at least one object of a plurality of objects of a study associated with a patient in a user interface;
identify a plurality of previous studies associated with the patient;
for each previous study of the plurality of previous studies, determine one or more findings associated with the previous study;
display at least one of the determined one or more findings in the user interface;
receive a selection of the displayed at least one of the determined one or more findings; and
in response to the selection, display the previous study associated with the selected at least one of the one or more determined findings in the user interface.

12. The system of claim 11, further comprising computer-executable instructions that when executed by the at least one computing device cause the system to:
in response to the selection, display one or more objects associated with the selected at least one of the one or more determined findings in the user interface.

13. The system of claim 11, wherein determining the one or more findings associated with the previous study comprises:
determining a report associated with the previous study;
retrieving a model trained to identify one or more findings based on a report; and
determining the one or more findings associated with the previous study using the model and the determined report.

14. The system of claim 13, further comprising computer-executable instructions that when executed by the at least one computing device cause the system to:
receive training data comprising a plurality of reports, wherein each report is labeled with at least one finding associated with the report; and
train the model using the training data by the computing device.

15. The system of claim 14, wherein the model comprises a machine learning model.

16. The system of claim 11, wherein the study comprises an image study.

17. The system of claim 11, wherein the objects comprise X-ray studies, CAT scan studies, ultrasound studies, positron emission tomography studies, nuclear medicine studies, mammography studies, or MRI studies.

18. The system of claim 11, wherein the one or more findings comprise one or more diagnoses.

19. The system of claim 11, wherein the at least one object is displayed in response to receiving a request to open the study.

20. A computer-readable medium with computer-executable instructions stored thereon that when executed by at least one computing device cause a system to:
display at least one object of a plurality of objects of a study associated with a patient in a user interface;
identify a plurality of previous studies associated with the patient;
for each previous study of the plurality of previous studies, determine one or more findings associated with the previous study;
display at least one of the determined one or more findings in the user interface;
receive a selection of the displayed at least one of the determined one or more findings; and
in response to the selection, display the previous study associated with the selected at least one of the one or more determined findings in the user interface.
